(19) United States
(12) Patent Application Publication
(10) Pub. No.: US 2019/0300845 A1
(43) Pub. Date: Oct. 3, 2019

(54) SEQUENTIAL ADDITION OF MOLECULAR GERMINANTS TO BACTERIAL SPORES

(71) Applicant: NOVOZYMES BIOAG A/S, Bagsvaerd (DK)

(72) Inventor: Jared Heffron, Salem, VA (US)

(73) Assignee: NOVOZYMES BIOAG A/S, Bagsvaerd (DK)

(21) Appl. No.: 16/315,719

(22) PCT Filed: Jun. 20, 2017
(86) PCT No.: PCT/US2017/038295

§ 371 Date: Jan. 7, 2019

Related U.S. Application Data
(60) Provisional application No. 62/359,759, filed on Jul. 8, 2016.

(51) Int. Cl. C12N 3/00; C12N 1/20; C12N 1/38

(52) U.S. Cl. CPC C12N 3/00; C12N 1/20; C12N 1/38; C12N 2500/40; C12N 2500/34; C12N 2500/38; C12N 2500/32

(57) ABSTRACT

We found that bacterial spores could be pretreated or initially treated with a partial complement of germinants (i.e., less germinants than would cause germination) and, subsequently, could be treated with the remaining germinants to cause germination of the spores. In some instances, the pre-treated spores germinated more efficiently than bacterial spores to which the full complement of germinants was simultaneously added to the spores.

### BACKGROUND

Germination of bacterial spores (e.g., endospores) to vegetative cells can be induced by contacting the spores with germinant molecules. These germinant molecules may be classed as nutrient germinants or non-nutrient germinants. Nutrient germinants may include, for example, substances like amino acids, sugars, purine nucleosides, or salts. Non-nutrient germinants may include lysozyme, dodecylamine, calcium dipicolinate, and others.

The specific molecules, how many different molecules are needed to cause germination, and the concentrations of the molecules needed for germination are variable and largely dependent on the bacterial strain that produced the spores. Spores from some bacterial strains, for example, may germinate in presence of a single amino acid, while spores from other strains may germinate in presence of specific combinations of amino acids, sugars, and salts. Knowledge of the molecules that cause germination of bacterial spores may facilitate use of bacterial spores in desired environments.

### SUMMARY

Germination of bacterial spores normally occurs when sufficient concentrations of the molecules needed for germination (i.e., germinants) are simultaneously contacted with the spores. We found that germinants could be contacted with bacterial spores sequentially, rather than simultaneously, and that germination still occurred. In some instances, sequential addition of germinants to bacterial spores resulted in more efficient germination of the spores, as determined by measurement of a variety of germination parameters, as compared to germination of spores caused by simultaneous addition of the germinants to the spores.

For example, for bacterial spores from a strain of Bacillus where a combination of a salt, an amino acid, and a sugar caused germination of the spores, we showed that the salt could be added to the spores first, without germination of the spores, and that the amino acid and sugar could subsequently be added to the spores to cause germination. Under some conditions, we found that the population of bacterial spores pretreated with the salt germinated more efficiently (e.g., a greater percentage of the spores in the population germinated) as compared to spores in a population that had not been pretreated with the salt.

In these studies, we found that the full complement of germinants generally needed to be present at the same time in order for germination to occur. For example, in the above example, if the salt added to the spores in the pretreatment step was subsequently removed from the spores (e.g., washed out), before the amino acid and sugar were added, germination did not generally occur.

These findings may facilitate use of bacterial spore-based compositions in certain environments. For example, bacterial spores may be used in an environment that lacks a molecule causing germination of the spores if the composition of spores to be dispersed into the environment contains the missing molecule. Spores in such a composition may actually germinate more efficiently when dispersed into the environment than they would if the molecule were present in the environment and not part of the composition containing the spores.

Disclosed herein are compositions, methods, and kits related to sequential addition of molecular germinants to bacterial spores.

### BRIEF DESCRIPTION OF THE DRAWINGS

In the accompanying drawings, which are incorporated in and constitute a part of the specification, embodiments of compositions, methods, and kits related to sequential addition of molecular germinants to bacterial spores are illustrated which, together with the detailed description given below, serve to describe the examples. It will be appreciated that the embodiments illustrated in the drawings are shown for the purpose of illustration and not for limitation. It will be appreciated that changes, modifications and deviations from the embodiments illustrated in the drawings may be made without departing from the spirit and scope of the invention, as disclosed below.

FIG. 1 A-C illustrates example spore germination data. (A) shows example germination kinetics, as determined from measurement of dipicolinic acid release from spores. T_{lag}, Gₘₐₓ, germination heterogeneity, and Gᵣₐₜₑ are shown. In (B), the dotted line shows more rapid initiation of germination (decreased T_{lag}), an increased rate of germination (increased Gᵣₐₜₑ), decreased germination heterogeneity, and increased number of spores that germinated (increased Gₘₐₓ), as compared to the solid line. In (C), the dotted line shows an increased Gₘₐₓ and increased germination heterogeneity, but no effect on T_{lag} or Gᵣₐₜₑ.

FIG. 2 illustrates example data from a spore germination experiment. Spores from Bacillus amyloliquefaciens strain SB3615 were used. Decrease in relative optical density (OD) indicates germination of spores. Spores were incubated in either brain-heart infusion medium (e), L-alanine (1), or buffer alone (+).

FIG. 3 illustrates example data from a spore germination experiment. Spores from Bacillus pumilis strain SB3189 were used. Decrease in relative optical density (OD) indicates germination of spores. Spores were incubated in either brain-heart infusion medium (●), L-alanine+D-fructose (▴), L-cysteine+D-fructose (▪), or buffer alone (+). The symbols representing L-alanine+D-fructose (▴) obscure the symbols for L-alanine+sucrose (traces are nearly the same). Likewise, the symbols for L-cysteine+D-fructose (▪) obscure the symbols for L-cysteine+sucrose (traces are nearly the same).

FIG. 4 illustrates example data from a spore germination experiment. Spores from Bacillus subtilis strain SB3086 were used. Percent germination of spores is indicated on the y-axis. Spores were germinated with D-fructose after initial incubation with another molecule. Spores were incubated initially in L-alanine (●), incubated in L-alanine and the L-alanine then washed out of the system before addition of D-fructose (▴), or were not incubated in L-alanine before addition of D-fructose (▪).

FIG. 5 illustrates example data from a spore germination experiment. Spores from Bacillus pumilis strain SB3189 were used. Percent germination of spores is indicated on the y-axis. Spores were germinated in 10 mM fructose after initial incubation with L-alanine at concentrations of 0 (+), 0.5 (□), 1 (Δ), 2, (x), 3 ( [symbol] ), 4 (∘), 5 (+), 6 ( [symbol] ) 7 (⋄), 8 (●), 9 (□), or 10 (▴) mM.

### DETAILED DESCRIPTION

### Definitions

The following includes definitions of selected terms that may be used throughout the disclosure and in the claims. The definitions include various examples and/or forms of components that fall within the scope of a term and that may be used for implementation. The examples are not intended to be limiting. Both singular and plural forms of terms fall within the definitions.

As used herein, “able to germinate,” in reference to bacterial spores, means that at least some of the spores in a population will germinate when provided with sufficient germinants.

As used herein, “about” means±10% with respect to the stated value or property.

As used herein, “add” means to put something together with something else.

As used herein, “after” means following.

As used herein, “alone” generally refers to whether germinants can cause germination of bacterial spores without the presence of other germinants. Germinants that alone can cause germination can cause germination without other germinants. Germinants that alone cannot cause germination, cannot, at least at a specific concentration, cause germination without other germinants.

As used herein, “bacteria” means prokaryotic organisms that have peptidoglycan in their cell walls, and have lipids that contain fatty acids in their membranes.

As used herein, “bacterial spores” refers to the structures formed by some bacteria during a process called sporulation. Generally, bacterial spores are resistant to environmental conditions, metabolically inactive, and unable to reproduce. Bacterial spores are generally able to germinate into vegetative cells.

As used herein, “capable of” refers to the ability or capacity to do or achieve a specific thing (e.g., ability of spores to germinate).

As used herein, “cause,” when used as a verb, means to make something happen.

As used herein, “combination” means things that are in proximity to one another or used together. For example, when a first germinant is in combination with a second germinant, the first and second germinants are in proximity to one another or used together.

As used herein, “compared to” means measurement of similarity or dissimilarity between things.

As used herein, “concentration” means an amount of something in a given volume.

As used herein, “contacting” means an act to cause things to physically touch. As used herein, “contact,” with reference to two or more things, means that the things physically touch each other.

As used herein, “contain” means to have or hold something within.

As used herein, “decrease” means to make or become smaller, fewer, or less.

As used herein, “different” means not the same as.

As used herein, “disperse” means to distribute or spread over an area.

As used herein, “dry” means free from liquid or moisture. Generally, a thing may be classified as dry based on moisture content.

As used herein, “efficiency,” may be used to describe germination of one population of spores as compared to a second population of spores. In some examples, a first population of germinating spores may be said to germinate with higher efficiency or more efficiently than a second population of germinating spores if, for example, the first population has a decreased T_{lag}, decreased germination heterogeneity, increased Gₘₐₓ, or increased Gᵣₐₜₑ as compared to the second population.

As used herein, “endospore” means a type of spore that develops inside of bacteria.

As used herein, “environment” means a particular physical location and/or set of conditions.

As used herein, “form,” when used as a verb, means to create something.

As used herein, “from” means the source of something.

As used herein, “germinant” means molecules that, alone or in combination with other germinants, generally at specific concentrations, have the ability to cause bacterial spores to germinate. Herein, water is not considered a germinant (i.e., the term “germinant” does not encompass water). A set of one or more germinants, at specific concentrations, that can cause germination of a population of bacterial spores, may be said to be a full complement or complete set of germinants. A set of one or more germinants, at specific concentrations, that do not cause germination of a population of bacterial spores, but that generally do cause germination in combination with one or more other germinants, may be said to be a partial complement or incomplete set of germinants.

As used herein, “germinate” refers to the process whereby a bacterial spore becomes a vegetative cell.

As used herein, “germination heterogeneity” refers to the window of time over which a population of spores germinates after receiving a stimulus sufficient to cause germination. Generally, this period of time begins when the T_{lag} period ends, and ends when the Gₘₐₓ is first reached. Germination heterogeneity is a germination parameter.

As used herein, “germination parameter” refers to a measurable factor describing germination of a population of bacterial spores. Example germination parameters include Gₘₐₓ, T_{lag}, germination heterogeneity, and Gᵣₐₜₑ.

As used herein, “Gₘₐₓ” refers to the percentage of bacterial spores within a population of spores that germinate. Gₘₐₓ is a germination parameter.

As used herein, “Gᵣₐₜₑ” refers to the rate at which spores germinate to vegetative cells and is generally visualized as the slope of the linear part of a germination curve (i.e., plot of germination over time). Gᵣₐₜₑ is a germination parameter

As used herein, “gram-positive” refers to bacteria that stain a certain way in a Gram stain procedure. Generally, gram-positive bacteria differ in their structure and/or arrangement of cellular membrane and cell wall as compared to gram-negative bacteria.

As used herein, “heat activate” refers to treatment of bacterial spores at a specific temperature for a specific period of time. Generally, bacterial spores are heat activated after a population of bacteria has substantially finished sporulating. In some examples, heat activation of spores may affect parameters of subsequent spore germination (e.g., increase efficiency of germination).

As used herein, “increase” means to make or become larger, greater, or more.

As used herein, “initial,” with reference to addition of germinants to bacterial spores, refers to one or more additions of germinants to bacterial spores that do not cause germination. Generally, germination may be caused by a “subsequent” addition of germinants to the spores.

As used herein, “kit” refers to a set or collection of two or more things, generally for use in a purpose. The two or more things that are part of a kit may be said to be “packaged” into or as a kit.

As used herein, “knowledge” means facts or information acquired by a person.

As used herein, “known” means recognized or within the scope of knowledge.

As used herein, “liquid,” refers to a state of matter that flows freely, has a definite volume and no fixed shape (e.g., it takes the shape of a container in which it is housed). Example liquids include, without limitation, emulsions, solutions, and suspensions.

As used herein, “mixture” means a combination of different things that are individually distinct. Herein, a mixture may be dry, moist, wet, or liquid.

As used herein, “moisture content” means the amount of water in a sample. Herein, moisture content is determined on a wet basis (i.e., mass of water in a sample/total mass of sample). For example, a sample with mass 10 grams, 1 gram of which is water, has a moisture content of 0.1 or 10%.

As used herein, “molecule” refers to two or more atoms held together by chemical bonds.

As used herein, “not present” means absent.

As used herein, “population” means a collection of things (e.g., bacterial spores) or totality of things in a group. In some examples a population of bacterial spores is called “stable.” The bacterial spores in a stable population generally are not undergoing germination, but may be capable of germinating or able to germinate.

As used herein, “possess” means to control or hold.

As used herein, “present” means to exist in a particular location.

As used herein, “prior” means before.

As used herein, “rate-limiting” generally refers to component that controls the outcome of a process. For example, a germinant may be said to be rate-limiting when a parameter of germination (e.g., Gₘₐₓ) is proportional to the concentration of the germinant.

As used herein, “set” means a group or collection of things. In some examples, a set of germinants may contain 1 or more germinants.

As used herein, “simultaneous” means at the same time.

As used herein, “single” means one.

As used herein, “solid,” refers to a state of matter that possesses structural rigidity and resistance to changes in shape or volume. Example solids include, without limitation, crystals, dusts, granules, gels, pastes, pellets, pressings, powders, and tablets.

As used herein, “specific” means particular or clearly identified.

As used herein, “subsequent,” with reference to addition of germinants to bacterial spores, refers to an addition of germinants to bacterial spores that occurs after one or more “initial” additions of germinants to the spores. Generally, the subsequent addition of germinants causes germination.

As used herein, “substance” means a particular thing with uniform properties.

As used herein, “sufficient” means enough or adequate.

As used herein, “T_{lag}” means the duration between the time when a population of bacterial spores receives a stimulus sufficient to cause germination and the time when spores in the population begin to germinate. T_{lag} is a germination parameter.

As used herein, “vegetative cells” refers to bacterial cells that are metabolically active and/or actively growing/dividing. Vegetative bacterial cells are not spores.

As used herein, “with” means accompanied by.

### Bacterial Spores

Some gram-positive bacteria may form bacterial spores or endospores under certain conditions. An example condition under which vegetative cells of bacteria form spores may be limiting amounts of nutrients needed for vegetative growth of the bacteria. Methods for obtaining bacterial spores from vegetative cells are well known in the field. In some examples, vegetative bacterial cells are grown in liquid medium. Beginning in the late logarithmic growth phase or early stationary growth phase, the bacteria may begin to sporulate. When the bacteria have finished sporulating, the spores may be obtained from the medium, by using centrifugation for example. Various methods may be used to kill or remove any remaining vegetative cells. Various methods may be used to purify the spores from cellular debris and/or other materials or substances. Some example methods for producing bacterial spores are described in Example 1 herein. Bacterial spores may be differentiated from vegetative cells using a variety of techniques, like phase-contrast microscopy or tolerance to heat, for example.

Bacterial spores are generally environmentally-tolerant structures that are metabolically inert or dormant. Sometimes, because of their environmental tolerance, bacterial spores are chosen to be used in commercial microbial products. These products may be designed to be dispersed into an environment where the spores will germinate and perform an intended function.

A variety of different bacteria may form spores. Bacteria from any of these groups may be used in the compositions, methods, and kits disclosed herein. For example, some bacteria of the following genera may form endospores: Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and/or Vulcanobacillus.

In some examples, the bacteria that may form endospores are from the genus Bacillus. In various examples, the Bacillus bacteria may be strains of Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, or combinations thereof.

In some examples, the bacterial strains that form spores may be strains of Bacillus, including: Bacillus pumilus strain NRRL B-50016; Bacillus amyloliquefaciens strain NRRL B-50017; Bacillus amyloliquefaciens strain PTA-7792 (previously classified as Bacillus atrophaeus); Bacillus amyloliquefaciens strain PTA-7543 (previously classified as Bacillus atrophaeus); Bacillus amyloliquefaciens strain NRRL B-50018; Bacillus amyloliquefaciens strain PTA-7541; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136 (also known as DA-33R, ATCC accession No. 55406); Bacillus amyloliquefaciens strain NRRL B-50141; Bacillus amyloliquefaciens strain NRRL B-50399; Bacillus licheniformis strain NRRL B-50014; Bacillus licheniformis strain NRRL B-50015; Bacillus amyloliquefaciens strain NRRL B-50607; Bacillus subtilis strain NRRL B-50147 (also known as 300R); Bacillus amyloliquefaciens strain NRRL B-50150; Bacillus amyloliquefaciens strain NRRL B-50154; Bacillus megaterium PTA-3142; Bacillus amyloliquefaciens strain ATCC accession No. 55405 (also known as 300); Bacillus amyloliquefaciens strain ATCC accession No. 55407 (also known as PMX); Bacillus pumilus NRRL B-50398 (also known as ATCC 700385, PMX-1, and NRRL B-50255); Bacillus cereus ATCC accession No. 700386; Bacillus thuringiensis ATCC accession No. 700387 (all of the above strains are available from Novozymes, Inc., USA); Bacillus amyloliquefaciens FZB24 (e.g., isolates NRRL B-50304 and NRRL B-50349 TAEGRO® from Novozymes), Bacillus subtilis (e.g., isolate NRRL B-21661 in RHAPSODY®, SERENADE® MAX and SERENADE® ASO from Bayer CropScience), Bacillus pumilus (e.g., isolate NRRL B-50349 from Bayer CropScience), Bacillus amyloliquefaciens TrigoCor (also known as “TrigoCor 1448”; e.g., isolate Embrapa Trigo Accession No. 144/88.4Lev, Cornell Accession No. Pma007BR-97, and ATCC accession No. 202152, from Cornell University, USA) and combinations thereof.

In some examples, the bacterial strains that form spores may be strains of Bacillus amyloliquefaciens. For example, the strains may be Bacillus amyloliquefaciens strain PTA-7543 (previously classified as Bacillus atrophaeus), and/or Bacillus amyloliquefaciens strain NRRL B-50154, Bacillus amyloliquefaciens strain PTA-7543 (previously classified as Bacillus atrophaeus), Bacillus amyloliquefaciens strain NRRL B-50154, or from other Bacillus amyloliquefaciens organisms.

In some examples, the bacterial strains that form spores may be Brevibacillus spp., e.g., Brevibacillus brevis; Brevibacillus formosus; Brevibacillus laterosporus; or Brevibacillus parabrevis, or combinations thereof.

In some examples, the bacterial strains that form spores may be Paenibacillus spp., e.g., Paenibacillus alvei; Paenibacillus amylolyticus; Paenibacillus azotofixans; Paenibacillus cookii; Paenibacillus macerans; Paenibacillus polymyxa; Paenibacillus validus, or combinations thereof.

Bacterial spores used in the compositions, methods, and kits disclosed herein may or may not be heat activated. In some examples, the bacterial spores are not heat inactivated.

For the compositions, methods, and kits disclosed here, populations of bacterial spores are generally used. In some examples, a population of bacterial spores may include bacterial spores from a single strain of bacterium. In some examples, a population of bacterial spores may include bacterial spores from 2, 3, 4, 5, or more strains of bacteria. Generally, a population of bacterial spores contains a majority of spores and a minority of vegetative cells. In some examples, a population of bacterial spores does not contain vegetative cells. In some examples, a population of bacterial spores may contain less than about 1%, 2%, 3%, 4%, 5%, 6%, 7%, 8%, 9%, 10%, 15%, 20%, 25%, 30%, 40%, or 50% vegetative cells, where the percentage of bacterial spores is calculated as ((vegetative cells/(spores in population+vegetative cells in population))×100). Generally, populations of bacterial spores used in the disclosed compositions, methods, and kits, are stable (i.e., not undergoing germination), with at least some individual spores in the population capable of germinating.

### Germination of Spores

Once formed, bacterial spores can exist as spores indefinitely. However, if bacterial spores receive sufficient stimuli, they may germinate into vegetative cells. Such stimuli may be said to cause germination. Generally, the stimuli that cause germination of spores include substances, molecules for example, whose presence, and possibly concentration, may be sensed or detected by the spores. Some germinants, referred to as nutrient germinants, are sensed when they interact with receptors in the inner membrane of the spores. Other germinants, referred to as non-nutrient germinants, are sensed by spores independent of receptors. Generally, germinants contact bacterial spores to cause germination.

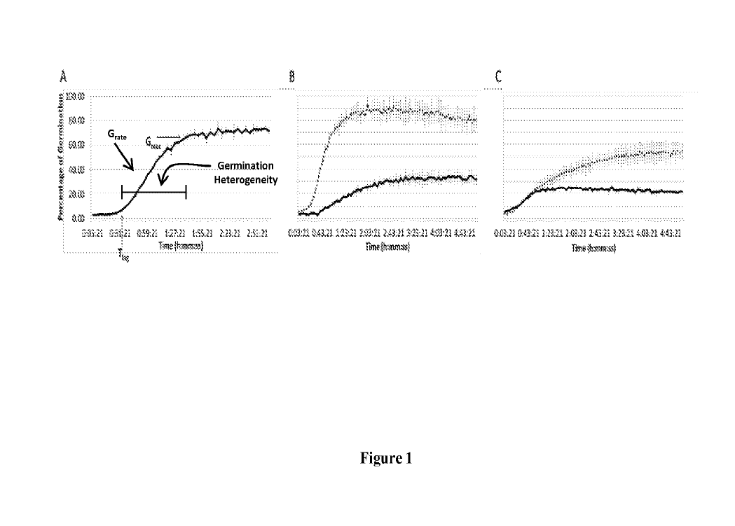

After a population of spores receives a stimulus sufficient to cause germination, germination of the population of spores may be heterogeneous. For example, individual spores within a spore population may germinate at different times after a stimulus sufficient to cause germination is received by the spores. The duration between the time that a sufficient stimulus occurs and the time when spores in a population begin to germinate is called T_{lag} (FIG. 1). The duration between the time when spores in the population begin to germinate and the time when spores cease to germinate is called germination heterogeneity (e.g., the window of time over which germination occurs; FIG. 1). The rate at which spores in the population germinate is called Gᵣₐₜₑ and is generally equivalent to the slope of the linear portion of a germination curve or plot (FIG. 1). Often, not all spores in a population will germinate after a stimulus sufficient to cause germination is received. The percentage of spores in a population that do germinate is called Gₘₐₓ (FIG. 1). All of these measurements—T_{lag}, germination heterogeneity, Gᵣₐₜₑ, and Gₘₐₓ—are parameters or characteristics that describe germination of the population of spores and are called germination parameters. Other germination parameters exist. A first population of spores that germinates with a decreased T_{lag}, decreased germination heterogeneity, increased Gᵣₐₜₑ, or increased Gₘₐₓ, as compared to a second population of spores may be said to germinate more efficiently than a second population of spores.

The process of germination may be measured or followed using a variety of methods. For example, bacterial spores appear shiny, bright, or refractile when viewed through a phase-contrast microscope, while vegetative bacterial cells appear dark or non-refractile. Bacterial spores release dipicolinic acid (DPA) when germination is caused. DPA release by spores can be measured. These methods are described and used in some of the studies described in the Examples of this application. Other methods for measuring germination of bacterial spores are known in the field and can be used.

### Germinants

A variety of events can cause bacterial spores to germinate. This disclosure generally concerns molecules that can cause germination. Generally, these molecules are called germinants. Germinants can, either alone or in combination with other germinants, cause germination of bacterial spores. The germinants may have to be present at certain concentrations in order to cause germination. Herein, when a germinant or set of germinants is said to “cause germination,” it means that the one or more germinants, when contacted with a population of spores, results in at least some of the spores in the population becoming vegetative bacterial cells. A single germinant that causes germination, or a combination of germinants that cause germination, may be said to be “sufficient” to cause germination, or may be referred to as a full complement or complete set of germinants. Single germinants or combinations of germinants that do not cause germination, may be referred to as partial complements or incomplete sets of germinants.

The molecules or combinations of molecules that cause germination of specific populations of spores may vary. For example, a single amino acid may cause spores from one species of bacteria to germinate, while an amino acid and a sugar, a sugar and a salt, or a sugar, salt, and an amino acid may be needed to cause germination of spores from another species. The molecules that cause germination of spores may be specific. For example, if an amino acid causes germination, it may be a specific amino acid. In other examples, the specificity may be less pronounced. For example, for some spores, if an amino acid causes germination, a number of amino acids may substitute for one another.

Spores from different strains of the same bacterial species may germinate under different conditions. For example, spores from one strain may need only L-alanine to germinate while spores from a second strain may need L-cysteine plus sucrose. Generally, the specific molecules or combination of molecules that cause germination are specific to a strain. Generally, the molecules that cause germination can be empirically determined.

Related to specificity and substitution of one germinant for another is the finding that spores from a single bacterium may have more than one germinant or combination of germinants that can cause germination. For example, spores from the same bacterium may germinate after contact with L-alanine plus D-fructose, L-histidine plus D-fructose, or L-leucine plus D-fructose. Some examples of this can be seen in Table 2 herein.

Germinants may have to contact bacterial spores at certain concentrations to cause germination. In some examples, a germinant may not cause germination when present below a certain concentration, but may cause germination above that concentration. In some examples, a germinant may cause germination when present below a certain concentration, but may not cause germination above that concentration. In some examples, too low of a germinant concentration, or too high of a germinant concentration may not cause germination—the germinant concentration may have to be within a range to cause germination.

In some examples, germinants may be present in compositions disclosed herein at concentrations of between about 0.001 mM-10.0 M, 0.01 mM-5.0 M, 0.1 mM-1.0 M, or 1.0 mM-0.1 M. In some examples, the germinants may be present in compositions disclosed herein at concentrations of about 0.01, 0.05, 1, 2, 3, 4, 5, 6, 7, 8, 10, 15, 20, 25, 30, 35, 40, 45, 50, 60, 70, 80, 90, or 100 mM. Concentrations of germinants may be selected such their addition to a population causes germination or does not cause germination.

Generally, the molecules that cause germination of a population of spores may be determined by testing. In some examples, various substances or combinations of substances may be added to a stable population of spores and a determination of whether germination occurs is made. In these tests, the environment in which the bacterial spores are placed may have an effect on the determination of germinants. For example, for a population of bacterial spores in a buffer that contains potassium, the testing may determine that a combination of L-alanine and D-glucose cause germination. However, if the same population of spores were in water, similar testing may determine that a combination of L-alanine, D-glucose, and KBr causes germination. Generally, there is at least some water present (e.g., an aqueous solution) for germination to occur. In the studies of this type described in Example 2 herein, the spores were in water when they were tested.

Some example molecules that may act as germinants, without limitation, include nutrient germinants or non-nutrient germinants. Example nutrient germinants may include amino acids, sugars, nucleosides, or salts. Example non-nutrient germinants may include lysozyme or other proteins, dodecylamine, calcium dipicolinate, and others.

Non-limiting examples of germinant molecules may include amino acids, salts, nucleosides, vitamins, and sugars. In some examples, amino acids may be L-amino acids. The amino acids may be classed in various ways. One method for classifying amino acids includes small amino acids (alanine, glycine), hydrophilic amino acids (cysteine, serine, threonine), hydrophobic amino acids (isoleucine, leucine, methionine, proline, valine), aromatic amino acids (phenylalanine, tryptophan, tyrosine), acidic amino acids (aspartic acid, glutamic acid), amide amino acids (asparagine, glutamine), and basic amino acids (arginine, histidine, lysine). Germinant molecules may include analogs of amino acids. Such analogs are known in the art.

In certain examples, some L-amino acids may be excluded from the subject matter encompassed by the term, germinants. In various examples, one or more of the following amino acids may be excluded: L-alanine, L-valine, L-proline, L-leucine, L-cysteine, L-threonine, L-glutamine, L-asparagine, or L-phenylalanine.

Example salts that are germinants, without limitation, may include KBr, KCl, MgSO₄, and NaCl. Non-limiting examples of purines/nucleosides may include adenine, adenosine, caffeine, guanine, guanosine, hypoxanthine, inosine, isoguanine, theobromine, uric acid, and xanthine. Non-limiting examples of vitamins may include β-alanine, biotin, folic acid, inositol, nicotinic acid, panthothenic acid, pyridoxine, riboflavin, and thiamine. Non-limiting examples of sugars may include arabinose, fructose, glucose, raffinose, and sucrose and lactose.

Non-limiting examples of germinants that may be suitable for the compositions, methods, and kits, described herein include lactate; lactose (as found in dairy products), bicarbonate or carbonate compounds such as sodium bicarbonate; carbon dioxide (e.g., carbonic acid: CO₂ dissolved in water, as is common in “sodas” or “soft drinks” such as cola or some fruit flavored beverages); compounds that adsorb lipid (e.g., starch, such as found in wheat, rice or other grains and potatoes and some other vegetables); charcoal or similar materials of high surface area that may adsorb or absorb fatty acid and lipid materials that may inhibit spore germination; monosaccharides such as fructose, glucose, mannose, or galactose; alanine, asparagine, cysteine, glutamine, norvatine, serine, threonine, valine, glycine, or other amino acid, and derivatives thereof such as N-(L-a-aspartyl)-L-phenylalanine (commonly sold under the trade name of “Aspartame”); inosine; bile salts such as taurocholate; and combinations of such spore germinants. For example, useful spore germinants can include alanine alone or in combination with lactate; a combination of L-asparagine, glucose, fructose, and potassium ion (AGFK); amino acids such as asparagine, cysteine, or serine alone or in combination with lactate; and caramels created by autoclaving monosaccharides or such caramels in combination with amino acids. In some examples, the composition comprises one or more germinants. In a particular embodiment, the composition comprises L-asparagine, glucose, fructose, and potassium ion (AGFK).

In this application, water (i.e., H₂O) is not considered to be a germinant. That is, when the term “germinant” is used herein, water is excluded from the meaning of the term.

### Sequential Addition of Germinants

In instances where sufficient germinants to cause germination of a population of bacterial spores are known, simultaneous addition of the full complement of germinants (i.e., all of the germinants, at the concentrations needed, to cause germination) to the population of bacterial spores generally causes germination of the spores.

Disclosed herein is the finding that simultaneous contacting of a population of bacterial spores with the full complement of germinants is not required to achieve germination. Instead, a partial complement (i.e., less than all of the germinants needed to cause germination; or all of the germinants, but at least one of the germinants at concentrations less than needed to cause germination) can initially be added to the population of bacterial spores with no or substantially no germination of the spores occurring. Subsequently, the remaining germinants needed to cause germination can be added to the population of bacterial spores, resulting in germination of the spores. In some instances, sequential addition of germinants to bacterial spores resulted in changed germination parameters of a population of bacterial spores, as compared to spores germinated by simultaneous addition of the full complement of germinants. In some instances, sequential addition of germinants caused more efficient spore germination than did simultaneous addition of germinants.

In some examples, to perform sequential addition of germinants, where multiple germinants are needed to cause germination of a population of bacterial spores, one or more of the multiple germinants may be added to the population of bacterial spores initially, as long as all of the germinants are not added to the spores (i.e., as long as at least one germinant needed for germination is omitted). For example, for a population of spores where two germinants are needed to cause germination, one of the germinants may be added initially, without occurrence of germination. The second germinant may be subsequently added to cause germination.

For a population of spores where three germinants are needed to cause germination, one of the germinants may be added initially, without occurrence of germination. The second and third germinants may be added subsequently to the spores to cause germination. Alternatively, two of the germinants may be added initially, without occurrence of germination. The third germinant may be subsequently added to the spores to cause germination. Generally, for sequential addition of germinants, when the initial germinants are added to the bacterial spores, there is no germination or there is substantially no germination of the spores. Germination occurs when the remaining germinants are added, subsequent to the germinants initially added to the spores.

As described earlier, germinants may have to be present at certain concentrations to cause germination of bacterial spores. The concentration requirements of germinants may be used in the compositions, methods, and kits related to sequential addition of germinants as disclosed herein. For example, consider a situation where multiple germinants, each present at a threshold concentration, or above a threshold concentration, are needed to cause germination of a population of bacterial spores. Using sequential addition of germinants, all of the needed multiple germinants may be added to the spores initially, without occurrence of germination, as long as at least one of the germinants is added at a concentration below its threshold needed to cause germination. More than one of the germinants may be added at a sub-threshold concentration. Under these conditions, no germination will occur. Germination occurs subsequently, when additional germinants are added to the spores such that all needed germinants are present at or above their threshold concentrations. Alternatively, less than all of the multiple germinants may be added initially to the bacterial spores, and one, more than one, or all of those may be present at sub-threshold concentrations, with no occurrence of germination.

For example, for a population of bacterial spores where one germinant is needed to cause germination, that germinant may be added to the spores initially, at a concentration below the threshold needed to cause germination, without germination of the spores. Subsequently, more of the germinant may be added to the spores, to raise the total concentration of the germinant at least to the threshold concentration, to cause germination.

For a population of spores where two germinants are needed to cause germination, both germinants may be added to the spores initially, without causing germination, as long as the concentration of at least one of the germinants is below the threshold needed for that germinant to cause germination. For example, the first of the two germinants may be present above its threshold concentration, while the second of the two germinants is present below its threshold concentration. Alternatively, the first of the two germinants may be present below its threshold concentration, while the second of the two germinants is present above its threshold concentration. Alternatively, both the first and the second of the needed germinants may be present below their threshold concentrations. Subsequently, additional amounts of the first, the second, or of both the first and the second germinants, may be added to the spores to increase the concentration of each needed germinant at least to its threshold, to cause germination.

One or more germinants added to bacterial spores at the same time may be referred to as a set of germinants. In some examples, a set of germinants may include a single germinant that is added to spores initially, without causing germination. In some examples, a set of germinants may include three germinants that are added to spores subsequently, and cause germination. Generally, unless indicated otherwise, a set of germinants may contain any number of germinants.

A first set of germinants may be said to be different from a second set of germinants if there is at least one difference between the germinants, or between germinant concentrations, in the two sets. Examples of sets of germinants that are different, include: i) a first set that contains L-alanine and a second set that contains glucose; ii) a first set that contains L-alanine and L-cysteine, and a second set that contains glucose and L-cysteine; or iii) a first set that contains 5 mM L-alanine and a second set that contains 10 mM L-alanine. Although sets of germinants used in the disclosed compositions, methods, and kits may be different from one another, the sets of germinants may be the same. That is, a set of germinants added to bacterial spores initially may be different or the same as a set of germinants added to the bacterial spores subsequently.

A set of germinants may be said to contain at least one germinant that is not present in another set of germinants. For example, a first set of germinants may contain glucose, while a second set may contain L-alanine and glucose. In this example, it may be said that at least one germinant (i.e., L-alanine) in the second set of germinants is not present in the first set of germinants.

When one or more germinants (i.e., partial complement of germinants) are said to be added to bacterial spores initially, the one or more germinants may be in contact with the spores for various periods of time before subsequent germinants are added to cause germination of the spores. In various examples, the germinants added initially may be in contact with the spores for about 1, 2, 3, 4, 5, 10, 15, 20, or 30 minutes; about 1, 2, 3, 4, 5, 6, 8, 10, 12, 15, or 18 hours; about 1, 2, 3, 4, 5, 10, 15, or 20 days; about 1, 2, 3, 4, 5, 6, 8, or 10 months; or about 1, 2, 3, 4, or 5 years; before subsequent germinants are added to cause germination of the spores.

Likewise, the duration between the initial addition of germinants and the subsequent addition of germinants may vary. In various examples, the subsequent addition or contacting of a population of bacterial spores with germinants may occur about 1, 2, 3, 4, 5, 10, 15, 20, or 30 minutes after; about 1, 2, 3, 4, 5, 6, 8, 10, 12, 15, or 18 hours after; about 1, 2, 3, 4, 5, 10, 15, or 20 days after, about 1, 2, 3, 4, 5, 6, 8, or 10 months after; or about 1, 2, 3, 4, or 5 years after the initial addition or contacting of the population of bacterial spores with germinants.

In some examples, bacterial spores and the one or more germinants initially added to the spores, may be kept at various temperatures for at least part of the time before subsequent germinants are added to cause germination of the spores. In various examples, the temperature may be 4° C., 5° C., 8° C., 10° C., 15° C., 20° C., 25° C., 30° C., 37° C., 42° C., 50° C., 60° C., 70° C., 80° C., or 90° C.

While a single “initial” addition of one or more germinants to bacterial spores is generally referred to, it may be that multiple “initial” additions of germinants to the bacterial spores may be used. These initial additions occur before a subsequent addition of germinants to the spores. The subsequent addition generally causes germination of the spores. For example, consider a population of bacterial spores for which an amino acid, a salt, and a sugar for germination to occur. In the context of multiple initial additions, the amino acid may be added to the bacterial spores first, without occurrence of germination. At some later time, the salt may be added to the bacterial spores, also without occurrence of germination. At a still later time, the sugar may be added to the bacterial spores, causing germination. In this example, amino acid addition may be considered an initial addition of a germinant to the spores. Addition of the salt may be considered a separate initial addition of a germinant to the spores. Addition of the sugar may be considered a “subsequent” addition. Addition of the sugar provides the final needed germinant and germination occurs.

In another example of multiple “initial” additions of germinants to spores, consider a population of bacterial spores where a threshold concentration of 40 mM of L-alanine causes germination. Here, 10 mM of L-alanine may be added to the bacterial spores first, without occurrence of germination. At a later time, an additional 10 mM of L-alanine may be added to the bacterial spores, also without occurrence of germination. At a still later time, an additional 10 mM of L-alanine may be added to the bacterial spores, again without occurrence of germination. Still later, an additional 10 mM of L-alanine may be added to the bacterial spores, this time causing germination. Of the four separate additions of L-alanine in this example, the first three additions may be considered “initial” additions and the last addition may be considered a “subsequent” addition.

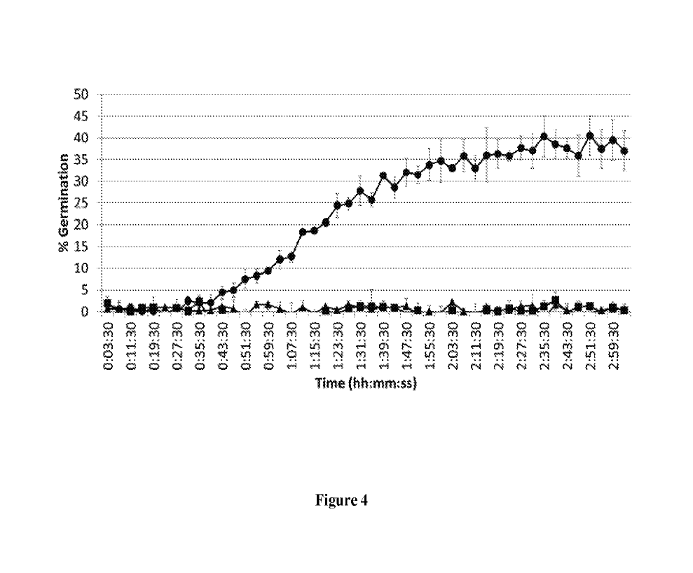

Generally, we have found that the germinants needed to cause germination of a population of spores need to be simultaneously present with the spores to cause germination. For example, for a population of bacterial spores that needs L-alanine and D-fructose to cause germination, we have found that L-alanine can be added initially and that D-fructose can be added subsequently to cause germination (Example 3 and FIG. 4). We have found that if L-alanine is added initially but then is removed from contact with the bacterial spores (e.g., by washing the spores in water) before D-fructose is added to the spores, that germination does not occur when the D-fructose is subsequently added. Therefore, that L-alanine was once in contact with the bacterial spores is not enough to provide for spore germination when D-fructose is subsequently added. Therefore, there does not appear to be spore “memory” that remembers that L-alanine was once in contact with the spores. Rather, the L-alanine and the D-fructose need to be present at the same time for germination to occur.

Whether germinants are added simultaneously or sequentially to a population of bacterial spores, water is generally present in order for germination of the spores to occur. In an example where a population of bacterial spores and a partial complement of germinants is present in an aqueous solution, subsequent addition of the remaining germinants may cause germination of the spores. However, where a population of bacterial spores and a partial complement of germinants is present in a non-aqueous form, subsequent addition of the remaining germinants, absent water, may not cause germination. Generally, water is present with the spores and the germinants for germination to occur.

Disclosed herein are compositions of bacterial spores and complements of germinants (e.g., bacterial spores and the germinants added initially, as described above). The bacterial spores in these compositions are stable in that they do not germinate until additional germinants are added to the compositions to provide a full complement of germinants (e.g., subsequent addition of germinants, as described above). These compositions may be, without limitation, in a solid state or a liquid state. In some examples, solid compositions may be made, without limitation, using techniques like spray drying, freeze drying, air drying, or drum drying. The solid compositions may be dry. In some examples, dry compositions may have a moisture content of less than about 50%, 40%, 30%, 25%, 20%, 15%, 12%, 10%, 9%, 8%, 7%, 6%, 5%, 4%, 3%, 2%, or 1%.

Regardless of the state of the compositions, the spores in these compositions are stable in that they are not germinating. However, they can germinate when provided with a full complement of germinants (generally water is also needed). There do exist in the art, stable dry compositions of bacterial spores that contain full complements of germinants (in one example, called “intimate mixtures” of spores and germinants). When water is added to these intimate mixture compositions, the bacterial spores in the compositions germinate. The solid compositions described herein—stable bacterial spores that contain a partial complement of germinants—which may be dry, generally do not germinate when water is added, because a full complement of germinants is not present.

### Effects on Germination Parameters

In some examples, sequential addition of molecular germinants to bacterial spores causes germination that is different than germination of the spores caused by simultaneous addition of the molecular germinants. In some instances, sequential addition of molecular germinants may result in more efficient germination of spores than simultaneous addition of germinants.

A variety of parameters of a population of germinating spores can be measured. T_{lag}, Gₘₐₓ, Gᵣₐₜₑ, and germination heterogeneity are example parameters of a germinating spore population that can be measured. Other parameters can be measured. The meaning of these particular terms and examples of how the parameters represented by these terms are determined can be found, for example, in the Definitions section and in FIG. 1, herein. In general, a first population of bacterial spores that has a decreased T_{lag}, increased Gₘₐₓ, increased Gᵣₐₜₑ, or decreased germination heterogeneity, as compared to a second population of bacterial spores, may be said to germinate more efficiently than the second population of bacterial spores. Sequential addition of germinants may cause germination that has one or more of these parameters of increased germination efficiency, as compared to germination of the same spores caused by simultaneous addition of the germinants. Parameters other those indicative of increased germination efficiency may be unchanged, or may change in a way indicative of less efficient germination, by sequential addition of the germinants.

In some examples, germinants may be said to be “known” to perform a function or achieve a result. In some examples, a composition of bacterial spores may contain a first germinant “known” not to cause germination of a spore population alone, but able to cause germination of the spore population in combination with a second “known” germinant. Recitation of “known” in these examples indicates that the composition was assembled with some knowledge, for example, of the molecules that cause germination of the particular spores used in the composition; of what the first and second germinants do, or don't do, to the particular spores; or how the first and second germinants function in combination.

In some examples, a method for germinating bacterial spores may recite contacting a bacterial spore population with a first germinant “known” to cause germination of the spore population in combination with a second germinant. Likewise, recitation of “known” in these examples may indicate that performance of the method contemplated the germination requirements of the bacterial spores used in the method; selection of the first and second germinants; etc.

### Uses of Sequential Germinant Addition

The compositions, methods, and kits disclosed herein may be used in a variety of circumstances. In some examples, a composition may be designed to contain bacterial spores because spores are known to be tolerant to a variety of adverse environmental conditions. Such a composition may have a longer shelf life and/or may better survive environmental insults than a product containing vegetative bacteria rather than the spores.

In some examples, the compositions, methods, and kits disclosed herein may be used in compositions to be applied to plants and/or plant leaves (i.e., agricultural use). In some examples, when so applied, the spores may germinate to vegetative bacterial cells which provide a useful function to plants. In some examples, the bacteria may provide biocontrol properties to the plant and/or enhance plant growth.

In some examples, the compositions, methods, and kits disclosed herein may be used in animal feed. Example bacterial spore-containing compositions may be mixed with animal feed or animal feed ingredients. This may be referred to as mash feed. In some examples, germination of the bacterial spores to vegetative bacteria in the mash feed may facilitate chemical breakdown of components of the mash. This may facilitate digestion of the mash feed in the animal digestive system or otherwise improve the digestive system of the animal.

In some examples, the compositions, methods, and kits disclosed herein may be used in detergents. The bacteria that produce the spores may be selected for inclusion in a detergent based on their ability to produce enzymes that may digest, for example, stains in a fabric. In some examples, deployment of the detergent may result in germination of the bacterial spores therein and production of the enzymes by the vegetative bacteria.

In some examples, compositions of a population of stable (i.e., not actively germinating) bacterial spores and one or more germinants may be dispersed into an environment. If the remaining germinants needed for germination of the spores are present in the environment, the spores may germinate in the environment. In some cases, the same bacterial spores in a composition that does not contain the one or more germinants may not germinate or may germinate less efficiently when dispersed into the environment.

### EXAMPLE EMBODIMENTS OF THE INVENTION

1. A first composition, comprising, consisting essentially of, or consisting of:

a population of bacterial spores and a first set of one or more germinants contacting the bacterial spores, the first set of germinants alone not sufficient to cause germination of the bacterial spores when in water;

the population of bacterial spores in the first composition able to germinate when contacted with a known second set of one or more germinants in water, the second set of germinants alone not sufficient to cause germination of the population of bacterial spores when in water.

2. The first composition of embodiment 1, where the composition is dry.

3. The first composition of embodiments 1 or 2, where the moisture content of the first composition is less than about 50%, 40%, 30%, 25%, 20%, 15%, 12%, 10%, 9%, 8%, 7%, 6%, 5%, 4%, 3%, 2%, or 1%.

4. The first composition of embodiment 1, where the composition is a liquid.

5. The first composition of any one of embodiments 1-4, where the germinants of the first set of one or more germinants are different than the germinants of the second set of one or more germinants.

6. The first composition of any one of embodiments 1-5, where at least one germinant in the second set of one or more germinants is not present in the first set of one or more germinants.

7. The first composition of any one of embodiments 1-6, where, when the second set of germinants is contacted with the population of bacterial spores in the first composition, and germination of the population of bacterial spores occurs, a parameter of the germination is different as compared to germination of the population of bacterial spores in a second composition that does not contain the first set of germinants, germination of the population of bacterial spores in the second composition occurring when the first and the second set of germinants are simultaneously contacted with the population of bacterial spores.

8. The first composition of embodiment 7, where the parameter of the germination that is different includes T_{lag}, germination heterogeneity, Gₘₐₓ, or Gᵣₐₜₑ.

9. The first composition of any one of embodiments 7 or 8, where the parameter of the germination that is different includes, a decrease in T_{lag}, a decrease in germination heterogeneity, an increase in Gₘₐₓ, or an increase in Gᵣₐₜₑ, in the first composition, as compared to the second composition.

10. The first composition of any one of embodiments 1-9, where the first set of one or more germinants contains one germinant.

11. The first composition of embodiment 10, where a concentration of the one germinant in the first set of germinants is rate-limiting for germination of the population of bacterial spores when the second set of germinants is contacted with the population of bacterial spores.

12. A first composition, comprising, consisting essentially of, or consisting of:
a population of bacterial spores and a single first germinant contacting the bacterial spores, the single first germinant present at a concentration that is not sufficient to cause germination of the population of bacterial spores when in water;
the population of bacterial spores in the first composition able to germinate when contacted with at least a second germinant in water;
where, when the population of bacterial spores in the first composition is contacted with the second germinant, and germination of the population of bacterial spores occurs, a parameter of the germination is different as compared to germination of the population of bacterial spores in a second composition that does not contain the first germinant, germination of the population of bacterial spores in the second composition occurring when a full complement of germinants is simultaneously contacted with the population of bacterial spores.

13. The first composition of embodiment 12, where the parameter of the germination that is different includes T_{lag}, germination heterogeneity, Gₘₐₓ, or Gᵣₐₜₑ.

14. The first composition of any one of embodiments 12 or 13, where the first germinant is different than the second germinant.

15. The first composition of any one of embodiments 12 or 13, where the first germinant is the same as the second germinant.

16. The first composition of embodiment 15, where the concentration of the first germinant is different than the concentration of the second germinant.

17. A composition, comprising, consisting essentially of, or consisting of:
a population of stable bacterial spores in contact with one or more first substances that are not water, the one or more first substances alone not causing germination of the population of stable bacterial spores when in water;
the one or more first substances known to cause germination of the population of stable bacterial spores when in water in combination with one or more second substances that are not water, the one or more second substances not present in the composition.

18. The composition of embodiment 17, where the composition is dry.

19. The composition of any one of embodiments 17 or 18, where the moisture content of the composition is less than about 50%, 40%, 30%, 25%, 20%, 15%, 12%, 10%, 9%, 8%, 7%, 6%, 5%, 4%, 3%, 2%, or 1%.

20. The composition of embodiment 17, where the composition is a liquid.

21. The composition of any one of embodiments 17-20, where the population of bacterial spores in the composition is capable of germinating with at least one of a decreased T_{lag}, increased Gₘₐₓ, decreased germination heterogeneity, or increased Gᵣₐₜₑ, when contacted with the one or more second substances in water, as compared to T_{lag}, Gₘₐₓ, germination heterogeneity, or Gᵣₐₜₑ of the population of bacterial spores in a composition that does not contain the one or more first substances, and are caused to geminate by contacting the population of bacterial spores with the one or more first substances at the same time as contacting the population of bacterial spores with the one or more second substances in water.

22. The composition of any one of embodiments 17-21, where the bacterial spores are from bacteria from the genera Acetonema, Actinomyces, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Coxiella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halobacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, or Seinonella.

23. The composition of any one of embodiments 17-22 where the bacterial spores are from the bacteria Bacillus amyloliquefaciens, Bacillus pumilis, or Bacillus subtilis.

24. The composition of any one of embodiments 17-23, where the bacterial spores are not heat activated.

25. The composition of any one of embodiments 17-24, where the one or more first substances include lactate, lactose, bicarbonate or carbonate compounds (e.g., sodium bicarbonate), carbon dioxide (e.g., carbonic acid, CO₂ dissolved in water), compounds that adsorb lipid (e.g., starch), charcoal or materials of high surface area that may adsorb or absorb fatty acid and lipid materials that may inhibit spore germination, monosaccharides (e.g., fructose, glucose, mannose, galactose), amino acids (e.g., alanine, asparagine, cysteine, glutamine, norvatine, serine, threonine, valine, glycine), amino acid derivatives (e.g., N-(L-a-aspartyl)-L-phenylalanine or “Aspartame”), inosine, or bile salts (e.g., taurocholate).

26. The composition of any one of embodiments 17-24, where the one or more first substances include at least one of an L-amino acid, salt, purine or nucleoside, vitamin, or sugar.

27. The composition of any one of embodiments 17-24, where the one or more first substances includes an L-amino acid or salt.

28. The composition of any one of embodiments 26 or 27, where the L-amino acid includes an amino acid from a subgroup of amino acids called small subgroup.

29. The composition of any one of embodiments 26-28, where the L-amino acid includes L-alanine.

30. The composition of any one of embodiments 26 or 27, where the salt includes a potassium salt.

31. The composition of any one of embodiments 26 or 27, where the salt includes KBr.

32. The composition of any one of embodiments 17-31, where the one or more first substances are present at a concentration of between about 0.001 mM-10.0 M, 0.01 mM-5.0 M, 0.1 mM-1.0 M, or 1.0 mM-0.1 M.

33. The composition of any one of embodiments 17-31, where the one or more first substances are present at a concentration of at least about 0.01, 0.05, 1, 2, 3, 4, 5, 6, 7, 8, 10, 15, 20, 25, 30, 35, 40, 45, 50, 60, 70, 80, 90, or 100 mM.

34. The composition of any one of embodiments 17-33, where the one or more first substances is one substance.

35. The composition of any one of embodiments 17-34, where the population of stable bacterial spores contains less than about 1%, 2%, 3%, 4%, 5%, 6%, 7%, 8%, 9%, 10%, 15%, 20%, 25%, 30%, 40%, or 50% vegetative cells.

36. A composition, comprising, consisting essentially of, or consisting of:
(a) a population of stable endospores, the stable endospores able to germinate when simultaneously contacted by specific amounts of at least n germinants in water, where n>1, the stable endospores not able to germinate when simultaneously contacted by less than the n germinants in water, or by up to n germinants in water, where at least one of the n germinants is present at less than the specific amounts;
(b) the composition also including at least 1 of the n germinants, but not more than n−1 of the n germinants, at the specific amounts, or by any number of the n germinants where at least 1 of the n germinants is present at less than the specific amounts, the germinants in the composition in contact with the stable endospores;
(c) where, when the stable endospores in the composition are contacted with the n germinants in water that are not in the composition, at the specific amounts, the stable endospores germinate with a decreased T_{lag}, increased Gₘₐₓ, decreased germination heterogeneity, or increased Gᵣₐₜₑ, as compared to germination of the population of stable endospores in a composition that does not contain the germinants of paragraph (b).

37. A method, comprising, consisting essentially of, or consisting of:
contacting a population of bacterial spores with amounts of one or more first substances that are not water that alone do not cause germination of the population of bacterial spores in water at the amounts, but that are known to cause germination of the population of bacterial spores in water in combination with amounts of one or more second substances, the population of bacterial spores and the one or more first substances forming a mixture.

38. The method of embodiment 37, where the mixture is dry.

39. The method of embodiments 37 or 38, where the moisture content of the mixture is less than about 50%, 40%, 30%, 25%, 20%, 15%, 12%, 10%, 9%, 8%, 7%, 6%, 5%, 4%, 3%, 2%, or 1%.

40. The method of embodiment 37, where the mixture is a liquid.

41. The method of any one of embodiments 37-40, where the population of bacterial spores in the mixture is capable of germinating with a decreased T_{lag}, increased Gₘₐₓ, decreased germination heterogeneity, or increased Gᵣₐₜₑ, as compared to the population of bacterial spores not in a mixture with the one or more first substances.

42. The method of any one of embodiments 37-41, where the one or more first substances alone would cause germination of the population of bacterial spores in water if present at a higher amount.

43. The method of any one of embodiments 37-41, where the one or more first substances alone would not cause germination of the population of bacterial spores in water if present at a higher amount.

44. The method of any one of embodiments 37-43, where the one or more first substances is one substance.

45. The method of any one of embodiments 37-44, where the one or more first substances includes an L-amino acid, salt, purine or nucleoside, vitamin, or sugar.

46. The method of any one of embodiments 37-45, where the one or more first substances includes an L-amino acid or salt.

47. The method of any one of embodiments 45 or 46, where the L-amino acid includes an amino acid from a subgroup of amino acids called small subgroup.

48. The method of any one of embodiments 45-47, where the L-amino acid includes L-alanine.

49. The method of any one of embodiments 45 or 46, where the salt includes a potassium salt.

50. The method of any one of embodiments 45 or 46, where the salt includes KBr.

51. The method of any one of embodiments 37-50, where the one or more first substances is present at a concentration of about 0.01, 0.05, 1, 2, 3, 4, 5, 6, 7, 8, 10, 15, 20, 25, 30, 35, 40, 45, 50, 60, 70, 80, 90, or 100 mM.

52. The method of any one of embodiments 37-51, where the population of bacterial spores within the mixture includes bacterial spores from the genus Bacillus.

53. The method of any one embodiments 37-52, where the population of bacterial spores within the mixture includes bacterial spores from Bacillus amyloliquefaciens, Bacillus pumilis, or Bacillus subtilis.

54. The method of any one of embodiments 37-53, where the population of bacterial spores in the mixture is not heat activated.

55. The method of any one of embodiments 37-54, including:

subsequently contacting the population of bacterial spores in the mixture with the one or more second substances to cause germination of the population of bacterial spores.

56. The method of embodiment 55, where the population of bacterial spores is contacted with the one or more second substances about 1, 2, 3, 4, 5, 10, 15, 20, or 30 minutes after; about 1, 2, 3, 4, 5, 6, 8, 10, 12, 15, or 18 hours after; about 1, 2, 3, 4, 5, 10, 15, or 20 days after, about 1, 2, 3, 4, 5, 6, 8, or 10 months after; or about 1, 2, 3, 4, or 5 years after being contacted with the one or more first substances.

57. The method of any one of embodiments 55 or 56, where, when the bacterial spores in the mixture germinate, a T_{lag} is decreased, a Gₘₐₓ is increased, a germination heterogeneity is decreased, or a Gᵣₐₜₑ is increased, as compared to germination of the population of bacterial spores when simultaneously contacted with the one or more first and the one or more second substances.

58. The mixture of any one of embodiments 37-57.

59. A method, comprising, consisting essentially of, or consisting of:
contacting a population of stable bacterial spores with one or more first germinants that can cause germination of the population of stable bacterial spores in water when in combination with one or more second germinants; and
subsequently contacting the population of stable bacterial spores and the one or more first germinants with the one or more second germinants to cause germination of the population of stable bacterial spores.

60. The method of embodiment 59, where germination of the population of stable bacterial spores occurs with a changed parameter as compared to germination of the population of stable bacterial spores not contacted with the one or more first germinants and not subsequently contacted with the one or more second germinants, but instead simultaneously contacted with the one or more first germinants and the one or more second germinants.

61. The method of embodiment 60, where the changed parameter includes T_{lag}, Gₘₐₓ, germination heterogeneity, or Gᵣₐₜₑ.

62. The method of embodiment 61, where T_{lag} is decreased, Gₘₐₓ is increased, germination heterogeneity is decreased, or Gᵣₐₜₑ is increased.

63. The method of any one of embodiments 59-62, where the subsequent contacting of the population of stable bacterial spores with the one or more second germinants occurs about 1, 2, 3, 4, 5, 10, 15, 20, or 30 minutes after; about 1, 2, 3, 4, 5, 6, 8, 10, 12, 15, or 18 hours after; about 1, 2, 3, 4, 5, 10, 15, or 20 days after, about 1, 2, 3, 4, 5, 6, 8, or 10 months after; or about 1, 2, 3, 4, or 5 years after the contacting of the population of stable bacterial spores with the one or more first germinants.

64. A method, comprising:
adding a first set of one or more substances that are not water, at specific amounts, to a population of bacterial spores, with knowledge that the first set of one or more substances, at the specific amounts, do not alone cause germination of the population of bacterial spores in water, but do cause germination of the population of bacterial spores in water in combination with a second set of one or more substances that are not water, added at specific amounts.

65. The method of embodiment 64, including subsequently adding the second set of one or more substances, at the specific amounts, to the population of bacterial spores to cause germination, the germination having a decreased T_{lag}, increased Gₘₐₓ, decreased germination heterogeneity, or increased Gᵣₐₜₑ, as compared to germination of the population of bacterial spores where germination is caused by simultaneously adding the first set of one or more substances and the second set of one or more substances, at the specific amounts, to the population of bacterial spores.

66. The method of any one of embodiments 64 or 65, where the second set of one or more substances, added above the specific amounts, do not alone cause germination of the population of bacterial spores in water.

67. A method, comprising, consisting essentially of, or consisting of:
possessing a composition of bacterial spores, the composition of bacterial spores including a first set of one or more germinants in contact with the bacterial spores, the first set of one or more germinants alone not sufficient to cause germination of the bacterial spores when in water, but known to cause germination of the bacterial spores when in water in combination with a second set of one or more germinants; and
dispersing the composition into an environment, where at least some of the spores germinate.

68. The method of embodiment 67, where the second set of one or more germinants is present in the environment.

69. The method of any one of embodiments 67 or 68, where the bacterial spores in the composition germinate more efficiently in the environment than would the bacterial spores in a composition that did not include the first set of one or more germinants.

70. A method, comprising, consisting essentially of, or consisting of:
dispersing into an environment, the first composition of any one of embodiments 1-16 or the composition of any one of embodiments 17-36.

71. A composition of any one of embodiments 1-16 or 17-36, for use in an environment.

72. A kit, comprising, consisting essentially of, or consisting of:

a population of bacterial spores, and

a set of one or more germinants, the germinants alone not sufficient to cause germination of the population of bacterial spores when in water;

the set of germinants designed to be added to the population of bacterial spores to form a mixture, prior to dispersal of the mixture into an environment.

73. The kit of embodiment 72, where the bacterial spores in the mixture are capable of germinating more efficiently in the environment than bacterial spores to which the set of germinants has not been added.

### EXAMPLES

The following examples are for the purpose of illustrating various embodiments and are not to be construed as limitations.

### Example 1. Preparation of Bacterial Spores

Bacteria from which spores were to be prepared were grown logarithmically in liquid culture. As carbon, nitrogen, and/or phosphorus in the logarithmic cultures became limiting (e.g., late in logarithmic growth), the vegetative cells began to sporulate. The cultures continued to be incubated until it was estimated that no additional spores would form in the cultures. In some cases, the spores were obtained from cultures that were production runs. The cultures were then centrifuged to pellet the spores, and remaining cells and debris. When these spore pellets were suspended in water, washed, again suspended in water, and the spore suspension allowed to settle in a tube, three visible layers generally formed. Microscopic examination of samples was used to confirm the presence of phase-bright spores at a desired purity (>99% phase-bright spores). If purity was not achieved, then water washing was repeated until desired purity was reached.

For the experiments described in Example 2, spores were prepared by subjecting the upper layer of settled spores to HistoDenz™ density gradient centrifugation. A 10-15 ml aliquot of the upper layer of settled spores was mixed with 20 ml of water in a tube. Larger cellular debris sedimented to the bottom of the tube while spores generally remained suspended in the water. The water containing the spores was transferred to a separate tube, while the cellular debris was left behind. The spores were centrifuged in a clinical centrifuge for 5 min at 8,000 rpm. The supernatant was discarded and the pellet was suspended in 25 ml of deionized water. After two additional washing steps, 10 ml of the spores were diluted with 20 ml of ice cold autoclaved water, and centrifuged for 5 min at 15,000 rpm. The spores formed a pellet which was suspended in ice cold autoclaved water. This centrifugation and suspension step was repeated two additional times. The spore pellet was then suspended in 5 ml of 20% HistoDenz™ (Sigma-Aldrich) in water (1 g HistoDenz™ in 5 ml of water). The spores in HistoDenz™ were layered on top of a 50% HistoDenz™ solution (5 g HistoDenz™ in 10 ml of water) in a 50 ml centrifuge tube. This tube was centrifuged for 35 min at 11,500 rpm. The bacterial spores formed a pellet at the bottom of the tube (vegetative cells and cellular debris formed a layer within the HistoDenz™ solution). The pellet was suspended in 5 ml of autoclaved water, diluted to a final volume of 50 ml and stored at 4° C. until needed.

### Example 2. Identifying Germinants

Studies were performed to determine germinants and/or germinant combinations sufficient for germination of spores obtained from various bacterial strains. Various amino acids, purines, nucleosides, sugars, salts, and vitamins were added to spores in water to determine whether these substances caused germination of the spores. The strategy was to first prepare and test combinations of these potential germinants. If a particular combination caused germination, subsequent combinations that contained a subset of the original combination were tested. Using this approach iteratively, it was generally possible to identify germinants that caused germination.

For example, a first combination of potential germinants containing 3 mM of all 20 essential L-amino acids was tested for ability to cause germination. If this combination caused germination, we concluded that one or more of the individual amino acids of the combination caused germination. Additional experiments were then performed using a second combination of amino acids, where one or more amino acids present in the first combination had been omitted (i.e., the second combination was a subset of the first combination). For example, amino acid solutions that omitted one or more specific of the amino acid subgroups indicated in Table 1 (e.g., small, hydrophilic, hydrophobic, aromatic, acidic, amide, basic) were prepared. If the spores germinated with the first combination, but did not germinate with the second combination, we concluded that one or more of the omitted components caused germination. Using this approach, we were able to ascertain germinants that caused germination of bacterial spores from a variety of strains.

TABLE 1
Substances tested for germinant activity

| Substance | Substance | |
|---|---|---|
| group | subgroup | Substance |
| Amino | Small | Alanine, Glycine |
| acids¹ | Hydrophilic | Cysteine, Serine, Threonine |
| | Hydrophobic | Isoleucine, Leucine, Methionine, Proline, |
| | | Valine |
| | Aromatic | Phenylalanine, Tryptophan, Tyrosine |
| | Acidic | Aspartic acid, Glutamic acid |
| | Amide | Asparagine, Glutamine |
| | Basic | Arginine, Histidine, Lysine |
| Salts | — | KBr, KCl |
| Purines/ | — | Adenine, Adenosine, Caffeine, Guanine, |
| nucleosides | | Guanosine, Hypoxanthine, Inosine, |
| | | Isoguanine, Theobromine, Uric acid, |
| | | Xanthine |
| Vitamins | — | β-alanine, Biotin, Folic acid, Inositol, |
| | | Nicotinic acid, Panthothenic acid, |
| | | Pyridoxine, Riboflavin, Thiamine |
| Sugars | — | Arabinose, D-fructose, D-glucose, raffinose, |
| | | sucrose |

¹Amino acids were L amino acids

To perform these studies, spores prepared as described in Example 1 were first heat activated in water at 68° C. for 30 min, cooled to room temperature, and suspended in 100 mM sodium phosphate buffer, pH 7.0, such that optical density of the suspension at 580 nm was 1.0 (OD₅₈₀=1.0). The heat-activated spores were checked for auto-germination by monitoring changes in light refraction for 20 min at 580 nm using a Tecan Infinite M200 96-well plate reader. Decreases in light refraction indicated spore germination occurred.

Spore preparations that did not auto-germinate were tested for ability to germinate under conditions where many substances with possible germinant activity were present by suspending the spores in brain heart infusion medium (BHI), a relatively rich medium. Spores that germinated in BHI medium were used in further experiments

The germinant testing experiments were performed in 96-well plates in a 200 μl final well volume (60 μl of the heat-activated spore stock in sodium phosphate buffer with OD₅₈₀=1.0, and 140 μl of germinant solution in water). Each potential germinant substance was present at a final concentration of 3 mM. The reaction mixtures were incubated for 70 minutes at 37° C. and OD₅₈₀ measurements were taken every minute during that time (decreased OD indicated germination). The readings were normalized by dividing each OD₅₈₀ reading by the reading obtained at time 0 (i.e., the time at which the germinant solution was added to the spores) to give relative OD₅₈₀ readings. Generally, germination of spore preparations, as shown by OD₅₈₀ readings, was confirmed using phase contrast microscopy and/or malachite green staining with a safranin counterstain (i.e., Schaeffer-Fulton method).

In the initial experiment for each spore preparation, the germinant solution contained all of the substances shown in Table 1 at a concentration of 3 mM (i.e., complete defined medium). If the spores germinated under these conditions, then subsequent iterative experiments used germinant solutions that lacked specific groups of germinants, as described above, to ascertain molecules that caused germination of spores from a specific bacterial strain.

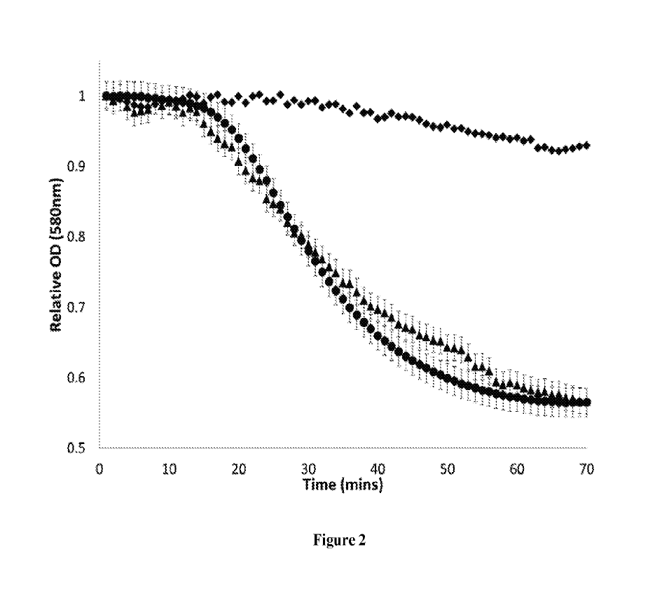

In some examples, spores from Bacillus amyloliquefaciens strain SB3615 were used in these experiments. SB3615 spores germinated in BHI medium (FIG. 2) and in complete defined medium (which contained all substances listed in Table 1 at 3 mM). Elimination of sugars, salts, vitamins, and purines/nucleosides had no effect on germination (i.e., these spores germinated in the presence of all 20 essential L-amino acids, without any other substances), which indicated that one or more amino acids caused germination of these spores. Therefore, we did germination experiments with each of the 20 essential L-amino acids alone. FIG. 2 shows data from one of these experiments, using L-alanine alone (3 mM concentration). The data indicate that the spores essentially germinated as well in the presence of L-alanine as they did in BHI medium (positive control). The spores did not germinate when no germinants were used (negative, buffer control). These data showed, that for spores from the SB3615 strain, that 3 mM L-alanine alone could cause germination.

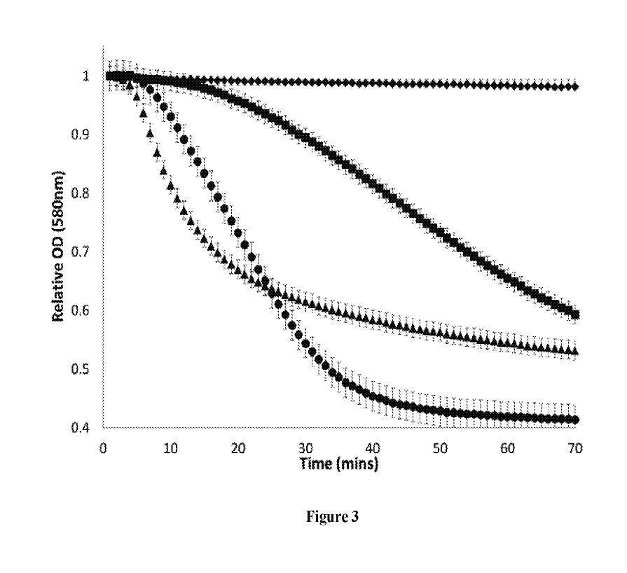

In another example, spores from Bacillus pumilus strain SB3189 were used in germination experiments. SB3189 spores germinated in BHI medium (FIG. 3) and in complete defined medium. Elimination of vitamins and purines/nucleosides had no effect on germination. These spores also germinated in a mixture of all 20 essential L-amino acids and sugars listed in Table 1, or all 20 essential L-amino acids and salts listed in Table 1. We tested each individual amino acid with individual sugars, and each individual amino acid with salts. From these experiments, we determined that combinations of L-alanine and D-fructose, or L-cysteine and D-fructose, all at 3 mM concentration, were sufficient to cause germination of these spores, as shown in FIG. 3.

We also found that sucrose could substitute for fructose, in combination with either L-alanine or L-cysteine. One possibility to explain the sucrose substitution was that sucrose degraded to glucose and fructose, and the released fructose acted as the germinant. We did not investigate this possibility. L-alanine in combination with either D-fructose or sucrose caused faster germination (lower T_{lag}) than did L-cysteine in combination with either fructose or sucrose. None of L-alanine, L-cysteine, D-fructose, or sucrose alone, at a concentration of 3 mM, caused germination.

In another example, spores from Bacillus megaterium strain SB3112 were used in germination experiments. SB3112 spores germinated in complete defined medium. Elimination of vitamins had no effect on germination. Since these spores could germinate in a mixture of L-amino acids, purines/nucleosides, sugars, we tested each group (i.e., amino acids, purines/nucleosides, sugars, salts) individually and in combinations of two of the groups, three or the groups, and four of the groups. From these experiments, we found six amino acids (L-alanine, L-histidine, L-isoleucine, L-leucine, L-phenylalanine, L-proline) that, along with KBr and D-glucose, caused germination of the spores.

The molecules/combinations of molecules that caused germination of spores from a variety of bacterial strains were determined using similar experiments. Table 2 shows the conclusions from these studies.

TABLE 2
Molecules causing germination of various bacterial spores

| Genus and species | | |
|---|---|---|
| of spores | Strain | Molecules causing germination¹ |
| Bacillus | SB3615 | L-alanine |
| amyloliquefaciens | | |
| Bacillus pumilus | SB3189 | L-alanine and D-fructose² |
| | | L-cysteine and D-fructose² |
| Bacillus subtilis | SB3086 | L-alanine and D-fructose |
| Bacillus megaterium | SB3112 | L-alanine, KBr, and D-glucose |
| | | L-histidine, KBr, and D-glucose |
| | | L-isoleucine, KBr, and D-glucose |
| | | L-leucine, KBr, and D-glucose |
| | | L-phenylalanine, KBr and D-glucose |
| | | L-proline, KBr, and D-glucose |

¹Each line entry indicates an independent germinant or germinant combination which causes germination
²Sucrose could substitute for D-fructose

### Example 3. Effects of L-Alanine Treatment on Germination of Bacillus subtilis Strain SB3086 Spores

The data in Table 2 indicate that a combination of 3 mM L-alanine and 3 mM D-fructose causes germination of spores from Bacillus subtilis strain SB3086. In this study, we investigated the timing of adding L-alanine relative to D-fructose (i.e., could L-alanine be added first, and D-fructose be added subsequently).

Bacterial spores from Bacillus subtilis strain SB3086 were prepared as described in the first paragraph of Example 1. The spore preparations were washed in sterile 4° C. water by centrifugation (10,000×g, 1 minute), aspirating the supernatant from the pellet, and suspending the spore pellet in water. This was performed three consecutive times. Optical densities of washed spore preparations were measured in sterile water at 4° C. using a Synergy H4 Multi-Mode Reader (Bio-Tek). The spore preparations were set to 5 OD₆₀₀ units per ml. By phase-contrast microscopy, 99% of the particles in the samples appeared to be ungerminated spores.

The spores were then treated with 1 mM of L-alanine for 24 hours at 4° C. No germination of the spores occurred as a result of the L-alanine treatment as determined by microscopy (germinated spores transition in appearance from phase-bright to phase-dark). In one experiment, spores that had been treated with 1 mM L-alanine for 24 hours were washed in water to remove the L-alanine. Negative controls were not treated with L-alanine. Spores to be used as positive controls were boiled for 2 hours to release dipicolinic acid (see below).

Germination of spores in these studies was determined by measuring dipicolinic acid (DPA). DPA is about 10% of the mass of endospores, which is generally released when germination occurs. Boiling spores generally releases all of the DPA (used as a positive control). DPA release was detected using terbium chloride (TbCl₃), as described below.

To perform the germination assays, 30 μl of spores were added to wells of a 96-well flat-bottom microtiter plate. Subsequently, 35 μl of a 250 μM TbCl₃ stock was added. At time 0, 85 μl of a D-fructose solution was added, to give a final concentration of 10 mM D-fructose. The solution was mixed by pipetting, the microtiter plate was placed into a plate reader (Synergy H4 Multi-Mode Reader, Bio-Tek), and the sample wells were measured for presence of a fluorescent Tb-DPA product over time. Tb-DPA is excited at 270 nm and emits at 545 nm. All experimental samples were tested in triplicate. Baseline controls for each sample were generated by omitting L-alanine and these were performed in duplicate. All sample wells were measured immediately after D-fructose was added (time 0) and then over time. For each time point, the data were normalized to values for the spores that had been boiled for two hours to force 100% release of DPA from the spores.

The data for an example experiment are shown in FIG. 4. FIG. 4 shows that SB3086 spores treated with 1 mM L-alanine for 24 hours germinated when 10 mM D-fructose was added, while spores not treated with L-alanine did not germinate with D-fructose.

These data show that L-alanine can be added to SB3086 spores initially, without causing germination, and that D-fructose can subsequently be added to cause germination. These data indicate that, for spores that require multiple substances for germination, that less than a full complement of germinants can be contacted with the spores without causing germination, and that the remaining germinants can subsequently be contacted with the spores to cause germination.

FIG. 4 shows that SB3086 spores that have been treated with L-alanine for 24 hours, but the L-alanine washed out of the system prior to addition of D-fructose, did not germinate when D-fructose was added. These data indicate that the separate molecules that cause germination need to be present with the spores at the same time in order to cause germination (i.e., germinants cannot be added and then removed prior to causing germination). We have shown this for a variety of different spores.

### Example 4. Effects of L-Alanine Treatment on Germination of Bacillus pumilus Strain SB3189 Spores

The data in Table 2 indicate the following combinations of components, each component present at 3 mM, cause germination of Bacillus pumilus strain SB3189 spores: L-alanine and D-fructose, L-alanine and sucrose, L-cysteine and D-fructose, and L-cysteine and sucrose. In this study, we investigated the effect of adding different concentrations of L-alanine to SB3189 spores prior to adding D-fructose.

The experiment was generally performed as described in Example 3. SB3189 spores were treated with various concentrations of L-alanine (0.0 mM, 0.5 mM, 1.0 mM, 2.0 mM, 3.0 mM, 4.0 mM, 5.0 mM, 6.0 mM, 7.0 mM, 8.0 mM, 9.0 mM, or 10.0 mM) for 24 hours at 4° C. After 24 hours, there was no germination of the spores due to the L-alanine treatment as determined by microscopy. D-fructose (10 mM) was then added to the L-alanine-treated spores (this was time 0) and DPA levels were measured over time.

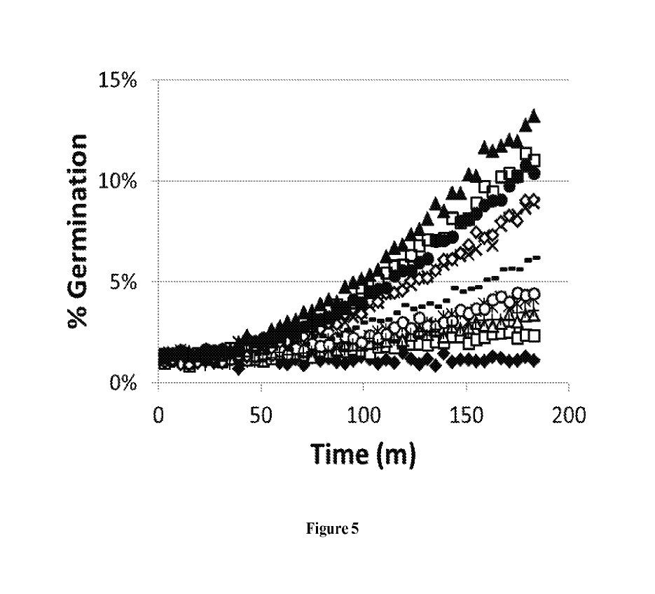

The data are shown in FIG. 5 and indicate that, for SB3189 spores treated with L-alanine, addition of D-fructose at time 0 caused germination of the spores (there was no germination in absence of L-alanine). The extent of germination increased as the amount of L-alanine increased. In this particular study, therefore, the concentration of L-alanine appeared to be rate-limiting for germination, at least up to 10 mM L-alanine used in this experiment.

Similar to the conclusions from the study shown in Example 3, these data support the conclusion that, for spores where multiple individual components cause germination, a less-than-full complement (i.e., partial complement) of the germinants can be added to the spores initially, without causing germination, and the remaining components can be added subsequently, to cause germination.

The data in FIG. 5 also show that spores not treated with L-alanine (indicated as 0 mM in FIG. 5) did not germinate in presence of D-fructose. These data are consistent with a combination of both L-alanine and D-fructose causing germination of SB3189 spores, as indicated in Table 2. These data are also consistent with the data in Example 3 showing that SB3086 spores (where L-alanine and D-fructose cause germination) didn't germinate when L-alanine was removed from the system prior to D-fructose addition.

### Example 5. Effects of KBr Treatment on Germination of Bacillus megaterium Strain SB3112 Spores

Table 2 indicates that spores from Bacillus megaterium strain SB3112 can be caused to germinate using multiple different combinations of molecules. These combinations include 3 components (i.e., an amino acid, a salt, a sugar). The salt in all of the combinations shown in Table 2 is KBr. The studies described below show that spore treatment with KBr, followed by treatment with a full complement of molecules sufficient for germination (i.e., the amino acid and the sugar), causes germination. In addition, germination parameters of SB3112 spores treated with KBr (notably Gₘₐₓ) were often different than the germination parameters of SB3112 spores not initially treated with KBr.

These studies were generally performed as described in Example 3. SB3112 spores, heat-activated or not heat-activated (not heat-activated are called “native”), were treated with 0.5, 3.0, or 10.0 mM KBr overnight at either 4° or 22° C. Subsequently, a solution was added to give a final concentration of 20 mM D-glucose, 20 mM L-proline, 20 mM L-asparagine, 20 mM L-valine, and an additional 50 mM of KBr, (this subsequent addition was enough to cause germination of SB3112 spores by itself; see Table 2). Addition of this solution was sufficient to cause germination of the spores, even in absence of the initial KBr treatment, as shown in the tables below. The time at which the subsequent addition was made to the spores was considered time 0. DPA concentration was measured over time.

The data from this study are shown in Tables 3 and 4, below. To facilitate comparison of the different samples, values for T_{lag} and Gₘₐₓ were calculated for each spore treatment. T_{lag} is the time after exposure to germinants that a population of spores begins to rapidly release DPA into the environment. T_{lag} indicates, in part, how rapidly a population of spores responds to germinants. Gₘₐₓ is the maximum percentage of DPA that a sample releases during an experiment, divided by the DPA released by the sample when boiled. Gₘₐₓ is proportional to the percentage of spores in a population that germinates. Both T_{lag} and Gₘₐₓ are determined from plots similar to that shown in FIG. 4.

As a preliminary matter, note that the data in Tables 3 and 4 show that SB3112 spores germinated even when there was no pretreatment with KBr (e.g., Gₘₐₓ of 22% at 0 mM KBr in Table 3; Gₘₐₓ of 61% at 0 mM KBr in Table 4). This is because, as discussed above, the solution added at time 0 to initiate germination included all of the molecules needed to germinate SB3189 spores (including 50 mM KBr). This is different than the experiments described in Examples 3 and 4, where the solution added to the spores at time 0 did not contain a full complement of the required germinants.

The data in Tables 3 and 4 indicate that initial contacting of spores with a partial molecular requirement for germination (i.e., KBr) can affect germination of the spores when subsequently contacted with the remaining molecules needed to cause germination. For example, the data show that incubation of native or heat-treated spores, at 4° C., with either 3.0 or 10.0 mM concentrations of KBr, increased the percentage of spores that germinated (i.e., Gₘₐₓ) as compared to spores not pretreated with KBr (Table 3). The same was found for incubation of native spores with KBr at 22° C. (Table 4).

However, for incubation of heat-treated spores, at 22° C., with 0.5, 3.0, or 10.0 mM concentrations of KBr, Gₘₐₓ was decreased as compared to spores not pretreated with KBr (Table 4). For some samples, T_{lag} for KBr-pretreated spores was also different as compared to spores not pretreated with KBr (e.g., 4° C. pretreatment of native spores with 3.0 or 10.0 mM KBr in Table 3; 22° C. pretreatment of heat-activated spores with 0.5 mM KBr in Table 4).

For SB3112 spores, the data showed that heat-activation of the spores may affect the ability of KBr to affect germination parameters.

TABLE 3
Effects of KBr treatment on germination of native and heat-activated
Bacillus megaterium strain SB3112 spores at 4° C.

| | |
|---|---|
| Native spores | Heat-activated spores |

| Concentration | T_{lag}¹ | Gₘₐₓ¹ | T_{lag}¹ | Gₘₐₓ¹ |
|---|---|---|---|---|
| of KBr (mM) | (min:sec) | (%) | (min:sec) | (%) |
| 0 | 23:30 | 22 | 7:30 | 90 |
| 0.5 | 23:30 | 24 | 7:30 | 92 |
| 3.0 | 19:30 | 31 | 7:30 | 123 |
| 10.0 | 19:30 | 29 | 7:30 | 85 |

¹Values are means of 3 replicates

TABLE 4
Effects of KBr treatment on germination of native and heat-activated
Bacillus megaterium strain SB3112 spores at 22° C.

| | |
|---|---|
| Native spores | Heat-activated spores |

| Concentration | T_{lag}¹ | Gₘₐₓ¹ | T_{lag}¹ | Gₘₐₓ¹ |
|---|---|---|---|---|
| of KBr (mM) | (min:sec) | (%) | (min:sec) | (%) |
| 0 | 7:30 | 61 | 7:30 | 83 |
| 0.5 | 7:30 | 62 | 11:30 | 65 |
| 3.0 | 7:30 | 96 | 7:30 | 65 |
| 10.0 | 7:30 | 103 | 7:30 | 61 |

¹Values are means of 3 replicates

These data indicate that, at least in some cases, spore pretreatment with partial complements of germinants affects parameters of spore germination when the spores are subsequently germinated, as compared to spores not initially treated with the partial complement of germinants.

While example compositions, methods, and so on have been illustrated by description, and while the descriptions are in considerable detail, it is not the intention of the applicants to restrict or in any way limit the scope of the application. It is, of course, not possible to describe every conceivable combination of components or methodologies for purposes of describing the compositions, methods, and so on described herein. Additional advantages and modifications will readily appear to those skilled in the art. Therefore, the invention is not limited to the specific details and illustrative examples shown and described. Thus, this application is intended to embrace alterations, modifications, and variations that fall within the scope of the application. Furthermore, the preceding description is not meant to limit the scope of the invention.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

1. A first composition, comprising:
a population of bacterial spores and a first set of one or more germinants contacting the bacterial spores, the first set of germinants alone not sufficient to cause germination of the bacterial spores when in water;
the population of bacterial spores in the first composition able to germinate when contacted with a known second set of one or more germinants in water, the second set of germinants alone not sufficient to cause germination of the population of bacterial spores when in water.

2. The first composition of claim 1, where the composition is dry.

3. The first composition of claim 1, where the composition is a liquid.

4. The first composition of claim 1, where, when the second set of germinants is contacted with the population of bacterial spores in the first composition, and germination of the population of bacterial spores occurs, a parameter of the germination is different as compared to germination of the population of bacterial spores in a second composition that does not contain the first set of germinants, germination of the population of bacterial spores in the second composition occurring when the first and the second set of germinants are simultaneously contacted with the population of bacterial spores.

5. The first composition of claim 4, where the parameter of the germination that is different includes, a decrease in T_{lag}, a decrease in germination heterogeneity, an increase in Gₘₐₓ, or an increase in Gᵣₐₜₑ, in the first composition, as compared to the second composition.

6. The first composition of claim 1, where the first set of one or more germinants contains one germinant.

7. The first composition of claim 6, where a concentration of the one germinant in the first set of germinants is rate-limiting for germination of the population of bacterial spores when the second set of germinants is contacted with the population of bacterial spores.

8. The composition of claim 1, where the bacterial spores are from bacteria from the genera Acetonema, Actinomyces, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Coxiella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halobacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, or Seinonella.

9. The composition of claim 1, where the bacterial spores are from the bacteria Bacillus amyloliquefaciens, Bacillus pumilis, or Bacillus subtilis.

10. The composition of claim 1, where the first set of one or more germinants include at least one of an L-amino acid, salt, purine or nucleoside, vitamin, or sugar.

11. The composition of claim 1, where the first set of one or more germinants are present at a concentration of at least about 0.01, 0.05, 1, 2, 3, 4, 5, 6, 7, 8, 10, 15, 20, 25, 30, 35, 40, 45, 50, 60, 70, 80, 90, or 100 mM.

12. A method, comprising:
contacting a population of bacterial spores with amounts of one or more first substances that are not water that alone do not cause germination of the population of bacterial spores in water at the amounts, but that are known to cause germination of the population of bacterial spores in water in combination with amounts of one or more second substances, the population of bacterial spores and the one or more first substances forming a mixture.

13. The method of claim 12, where the population of bacterial spores in the mixture is capable of germinating with a decreased T_{lag}, increased Gₘₐₓ, decreased germination heterogeneity, or increased Gᵣₐₜₑ, as compared to the population of bacterial spores not in a mixture with the one or more first substances.

14. The method of claim 12, where the one or more first substances alone would cause germination of the population of bacterial spores in water if present at a higher amount.

15. The method of claim 12, where the one or more first substances include an L-amino acid, salt, purine or nucleoside, vitamin, or sugar.

16. The method of claim 12, where the population of bacterial spores within the mixture includes bacterial spores from the genus Bacillus.

17. The method of claim 12, including:
subsequently contacting the population of bacterial spores in the mixture with the one or more second substances to cause germination of the population of bacterial spores.

18. The method of claim 17, where, when the bacterial spores in the mixture germinate, a T_{lag} is decreased, a Gₘₐₓ is increased, a germination heterogeneity is decreased, or a Gᵣₐₜₑ is increased, as compared to germination of the population of bacterial spores when simultaneously contacted with the one or more first and the one or more second substances.

19. The method of claim 17, where the subsequent contacting of the population of bacterial spores in the mixture with the one or more second substances occurs at least about 1 year after the contacting of the population of bacterial spores with the one or more first substances.

20. The method of claim 17, where the subsequent contacting of the population of bacterial spores in the mixture with the one or more second substances occurs at least about 6 months after the contacting of the population of bacterial spores with the one or more first substances.
